(19) United States
(12) Patent Application Publication
(10) Pub. No.: US 2025/0312185 A1
(43) Pub. Date: Oct. 9, 2025

(54) SIMULATION DEVICE AND METHOD FOR AN ARTICULATOR

(71) Applicant: SHARESMILE BIOTECH CO., LTD., New Taipei City (TW)

(72) Inventors: Jui-Hsiang SHEN, New Taipei City (TW); Yuan-Chieh CHENG, New Taipei City (TW); Yi-Jyun CHEN, New Taipei City (TW)

(21) Appl. No.: 18/974,690

(22) Filed: Dec. 9, 2024

(30) Foreign Application Priority Data
Apr. 3, 2024 (TW) 113112821

(51) Int. Cl. A61F 5/56; B29C 33/38; G06F 30/27

(52) U.S. Cl. CPC A61F 5/566; B29C 33/3835; G06F 30/27

(57) ABSTRACT

A simulation device for an articulator is disclosed. The simulation device includes a transceiver circuit, a memory, and a processor and is suitable for simulating the articulator in a three-dimensional virtual space. The processor of the simulation device performs following steps: obtaining multiple reference positions respectively corresponding to multiple occlusion states from multiple dentition three-dimensional model diagrams; generating a target position based on the multiple reference positions and multiple two-dimensional occlusion images; generating a new dentition three-dimensional model corresponding to a target occlusion state based on the target position, and establishing an articulator target model of the new dentition three-dimensional model diagram.

### BACKGROUND OF THE DISCLOSURE

### Technical Field

The present disclosure relates to simulation technology for positioning imaging, and more particularly to a simulation device and method for occlusal molds.

### Description of Related Art

In the current treatment techniques for sleep apnea, medical personnel often need to perform surgery on the patient's throat or nasal cavity. Such treatment methods require the patient to be under general anesthesia, and an endoscope is used to partially remove, reshape, or reconstruct tissues inside the throat or nasal cavity to increase the airway space. However, since such treatment methods require general anesthesia for the patient, there is a certain degree of medical risk. Additionally, after partially removing, reshaping, or reconstructing the tissues inside the throat or nasal cavity, some tissues may self-repair and cause the airway space to revert to its original state. Moreover, the patient may also experience a recurrence of sleep apnea due to muscle relaxation with age and weight gain. Therefore, how to avoid the medical risks associated with surgery and the reduced efficacy after surgery is a problem that technical personnel in this field urgently need to solve.

### SUMMARY OF THE INVENTION

The main objective of this disclosure is to provide a simulation device and method for occlusal molds, which can avoid the medical risks associated with surgery and the reduced efficacy after surgery.

To achieve the above objective, the simulation device for occlusal molds disclosed herein is suitable for simulating an occlusal mold in a three-dimensional virtual space, where the simulation device includes:
a transceiver circuit, configured to receive multiple three-dimensional dental model diagrams in the three-dimensional virtual space corresponding to multiple teeth occlusion states from a scanning and modeling device, and to receive multiple two-dimensional occlusion images corresponding to the multiple teeth occlusion states from an image forming device. The multiple teeth occlusion states include a maximum forward displacement state of the lower jaw, a maximum open state, and a natural occlusion state;
a memory, configured to store multiple instruction;
a processor, connected to the transceiver circuit and the memory, configured to execute the multiple instructions to perform the following steps:
obtain multiple reference positions from the multiple three-dimensional dental model diagrams corresponding to the multiple teeth occlusion states, where each of the multiple reference positions is a position of a dental arch center point of a lower jaw when a patient's mouth is in the teeth occlusion state corresponding to each of the multiple reference positions;
generate a target position based on the multiple reference positions and the multiple two-dimensional occlusion images, where the target position is a position of the dental arch center point of the lower jaw when the patient's mouth is in a target occlusion state, where the target occlusion state indicates that the patient's epiglottis and trachea are in a more open state; and
generate a new three-dimensional dental model diagram corresponding to the target occlusion state based on the target position and establish an occlusal target model of the new three-dimensional dental model diagram.

To achieve the above objective, the disclosed simulation method for occlusal molds is suitable for simulating an occlusal mold in a three-dimensional virtual space, where the simulation method includes:
using a processor to obtain multiple reference positions from the multiple three-dimensional dental model diagrams in the three-dimensional virtual space corresponding to the multiple teeth occlusion states, where each of the multiple reference positions is a position of a dental arch center point of a lower jaw when a patient's mouth is in the teeth occlusion state corresponding to each of the multiple reference positions. The multiple three-dimensional dental model diagrams and the multiple two-dimensional occlusion images correspond to multiple teeth occlusion states, including a maximum forward displacement state of the lower jaw, a maximum open state, and a natural occlusion state;
using the processor to generate a target position based on the multiple reference positions and the multiple two-dimensional occlusion images, where the target position is a position of the dental arch center point of the lower jaw when the patient's mouth is in a target occlusion state, where the target occlusion state indicates that the patient's epiglottis and trachea are in a more open state; and
using the processor to generate a new three-dimensional dental model diagram corresponding to the target occlusion state based on the target position and establish an occlusal target model of the new three-dimensional dental model diagram.

Compared to the prior art, the disclosure has the following advantages: it can accurately locate the target position, thereby obtaining an accurate occlusal target model to manufacture an occlusal mold that fits the patient's teeth, allowing the patient to wear such a mold with comfort, good occlusion, and smooth breathing. This avoids the medical risks of traditional surgical treatments and the reduced efficacy after surgery.

### BRIEF DESCRIPTION OF THE DRAWINGS

FIG. 1 illustrates a block diagram of a simulation device for occlusal molds in some embodiments of the present disclosure.

FIG. 2A illustrates a schematic diagram of a three-dimensional dental model in a natural occlusion state in some embodiments of the present disclosure.

FIG. 2B illustrates a schematic diagram of a three-dimensional dental model in a maximum open state in some embodiments of the present disclosure.

FIG. 2C illustrates a schematic diagram of a three-dimensional dental model in a maximum forward displacement state of the lower jaw in some embodiments of the present disclosure.

FIG. 3A illustrates a schematic diagram of a two-dimensional occlusion image in a natural occlusion state in some embodiments of the present disclosure.

FIG. 3B illustrates a schematic diagram of a two-dimensional occlusion image in a maximum open state in some embodiments of the present disclosure.

FIG. 4 illustrates a flowchart of the simulation method in some embodiments of the present disclosure.

FIG. 5A illustrates a schematic diagram of the dental arch center point of the lower jaw in a three-dimensional dental model in a natural occlusion state in some embodiments of the present disclosure.

FIG. 5B illustrates an enlarged side cross-sectional view of a part of the three-dimensional dental model in a natural occlusion state in some embodiments of the present disclosure.

FIG. 6A illustrates a schematic diagram of the dental arch center point of the lower jaw in a three-dimensional dental model in a maximum open state in some embodiments of the present disclosure.

FIG. 6B illustrates an enlarged side cross-sectional view of a part of the three-dimensional dental model in a maximum open state in some embodiments of the present disclosure.

FIG. 7A illustrates a schematic diagram of the dental arch center point of the lower jaw in a three-dimensional dental model in a maximum forward displacement state in some embodiments of the present disclosure.

FIG. 7B illustrates an enlarged side cross-sectional view of a part of the three-dimensional dental model in a maximum forward displacement state in some embodiments of the present disclosure.

FIG. 8A illustrates a schematic diagram of the opening in a two-dimensional occlusion image corresponding to a natural occlusion state in some embodiments of the present disclosure.

FIG. 8B illustrates a schematic diagram of the opening in a two-dimensional occlusion image corresponding to a maximum open state in some embodiments of the present disclosure.

FIG. 9A illustrates a schematic diagram of a three-dimensional dental model in a target occlusion state in some embodiments of the present disclosure.

FIG. 9B illustrates an enlarged side cross-sectional view of a part of the three-dimensional dental model in a target occlusion state in some embodiments of the present disclosure.

FIG. 10A illustrates a schematic diagram of an adjusted three-dimensional dental model in some embodiments of the present disclosure.

FIG. 10B illustrates a schematic diagram of an adjusted three-dimensional dental model in some other embodiments of the present disclosure.

FIG. 11A illustrates a schematic diagram of an occlusal target model in some embodiments of the present disclosure.

FIG. 11B illustrates a schematic diagram of the detailed structure of an occlusal target model in some embodiments of the present disclosure.

FIG. 12 illustrates a schematic diagram of a two-dimensional occlusion image taken of a patient wearing the occlusal mold in some embodiments of the present disclosure.

FIG. 13 illustrates a schematic diagram of the simulation outlet in some embodiments of the present disclosure.

### DETAILED DESCRIPTION

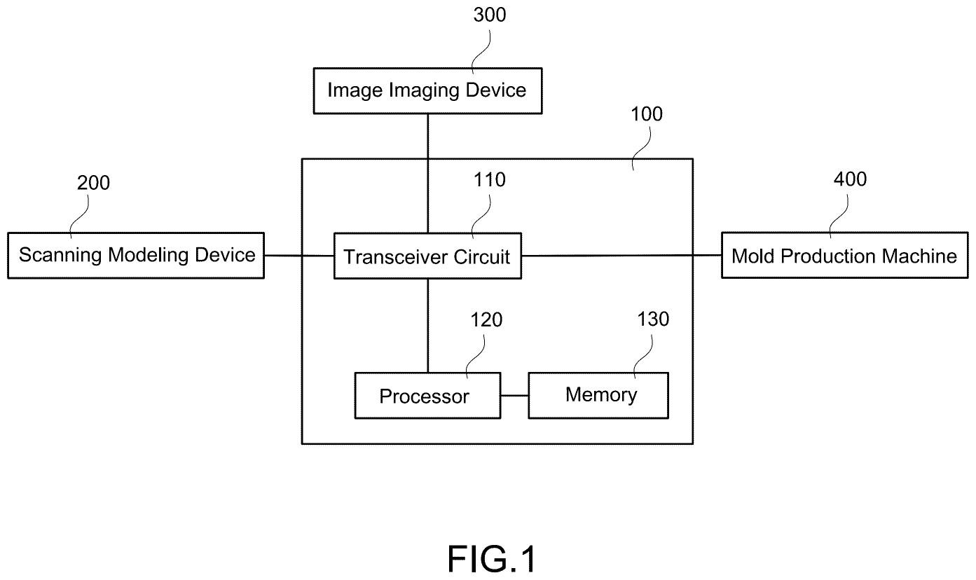

Referring to FIG. 1, FIG. 1 illustrates a block diagram of a simulation device 100 for an occlusal mold in some embodiments. The simulation device 100 for the occlusal mold shown in FIG. 1 can be implemented by any electronic device or server (e.g., a terminal processing device such as a smartphone, desktop computer, or tablet, a cloud device, server, or cloud server). The simulation device 100 is suitable for simulating the occlusal mold in a three-dimensional virtual space. By this means, the simulation device 100 can transmit the optimal simulated occlusal model to an external mold production machine 400 to produce a physical occlusal mold corresponding to the optimal occlusal model. The occlusal mold corresponding to the optimal occlusal model is used for patients to bite in order to treat obstructive sleep apnea.

Furthermore, when a patient bites down on the occlusal mold corresponding to the optimal occlusal model, the patient's epiglottis and trachea are in a more open state (i.e., open to an adequate and comfortable breathing state). In this way, as long as the patient bites this occlusal mold, it can increase the patient's airway space to prevent the occurrence of obstructive sleep apnea. By using the occlusal mold, the patient no longer needs general anesthesia for surgery, thereby addressing the medical risks associated with surgery and the reduced efficacy after surgery. On the other hand, when the patient uses the occlusal mold corresponding to the optimal occlusal model, the patient's mandible can bite the occlusal mold in a more comfortable posture, thus avoiding the issue of excessive mandibular muscle soreness caused by excessive force. The subsequent paragraphs will describe the simulation of the optimal occlusal model and the structure of the occlusal mold, so further details are not provided here.

As shown in FIG. 1, the simulation device 100 for the occlusal mold includes a transceiver circuit 110, a processor 120, and a memory 130. The processor 120 is connected to the transceiver circuit 110 and the memory 130.

In this embodiment, the transceiver circuit 110 receives multiple three-dimensional virtual space dental models corresponding to various dental occlusion states from a scanning modeling device 200, and then receives multiple two-dimensional occlusion images corresponding to the same dental occlusion states from an imaging device 300. The multiple dental occlusion states include at least the maximum forward movement state of the mandible, the maximum mouth-opening state, and the natural occlusion state.

In some embodiments, the transceiver circuit 110 may be one or a combination of a transmitter circuit, an analog-to-digital converter, a digital-to-analog converter, a low-noise amplifier, a mixer, a filter, an impedance matcher, a transmission line, a power amplifier, one or more antenna circuits, and a local storage media component. In some embodiments, the maximum forward movement state of the mandible refers to a state where the patient's mandible moves forward along a horizontal direction relative to the ground by the maximum horizontal distance (i.e., the limit distance the patient's mandible can move forward horizontally). In some embodiments, the maximum mouth-opening state refers to a state where the patient's mandible moves downward along a vertical direction relative to the ground by the maximum vertical distance (i.e., the limit distance the patient's mandible can move downward vertically). In some embodiments, the natural occlusion state refers to a state where the patient's mandible naturally bites the upper jaw.

In some embodiments, the multiple dental occlusion states may include other types of occlusion states. For example, the occlusion states may include a first occlusion state where the patient's mandible moves forward along a horizontal direction relative to the ground by half of the maximum horizontal distance and downward along a vertical direction relative to the ground by half of the maximum vertical distance, or a second occlusion state where the patient's mandible moves forward along a horizontal direction relative to the ground by one-third of the maximum horizontal distance and downward along a vertical direction relative to the ground by half of the maximum vertical distance.

In some embodiments, the multiple three-dimensional dental models in the virtual space are generated by scanning the patient's mouth in various tooth occlusion states using the scanning modeling device 200. In some embodiments, the multiple two-dimensional occlusion images are captured by the imaging device 300, depicting the patient's face and neck (e.g., from the entire profile to the throat) in various tooth occlusion states. In some embodiments, the multiple two-dimensional occlusion images may also include side views of the patient's face and neck in partial tooth occlusion states (e.g., maximum mouth opening state and natural occlusion state) captured by the imaging device 300

It is noteworthy that when using imaging devices 300 that produce high radiation levels (e.g., X-ray imaging), the imaging device 300 will capture only a few two-dimensional occlusion images (i.e., fewer two-dimensional occlusion images) to prevent excessive radiation absorption by the patient's body. Conversely, when using imaging devices 300 that do not produce high radiation levels (e.g., ultrasound imaging), the imaging device 300 can capture multiple two-dimensional occlusion images (i.e., a large number of two-dimensional occlusion images).

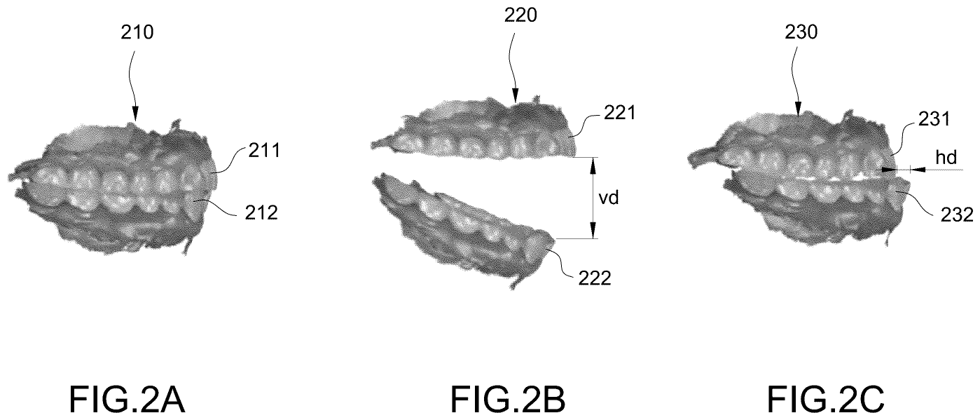

The following provides practical examples of dental models and two-dimensional occlusion images. Referring to FIGS. 2A-2C, FIG. 2A shows a schematic diagram of a dental three-dimensional model 210 in the natural occlusion state, FIG. 2B shows a schematic diagram of a dental three-dimensional model 220 in the maximum mouth-opening state, and FIG. 2C shows a schematic diagram of a dental three-dimensional model 230 in the maximum forward movement state.

As shown in FIG. 2A, the dental three-dimensional model 210 is a three-dimensional model generated by scanning the patient's mouth in the natural occlusion state. The dental three-dimensional model 210 includes an upper jaw model 211 and a lower jaw model 212. The lower jaw model 212 is a three-dimensional simulated model of the lower jaw in the natural occlusion state. The upper jaw model 211 is a three-dimensional simulated model of the unmovable upper jaw. As shown in FIG. 2B, the dental three-dimensional model 220 is a three-dimensional model generated by scanning the patient's mouth in the maximum mouth-opening state. The dental three-dimensional model 220 includes an upper jaw model 221 and a lower jaw model 222. The lower jaw model 222 is a three-dimensional simulated model of the patient's lower jaw moved downward by the maximum vertical distance vd. The upper jaw model 221 is also a three-dimensional simulated model of the unmovable upper jaw. As shown in FIG. 2C, the dental three-dimensional model 230 is a three-dimensional model generated by scanning the patient's mouth in the maximum forward movement state. The dental three-dimensional model 230 includes an upper jaw model 231 and a lower jaw model 232. The lower jaw model 232 is a three-dimensional simulated model of the patient's lower jaw moved forward by the maximum horizontal distance hd. The upper jaw model 231 is also a three-dimensional simulated model of the unmovable upper jaw.

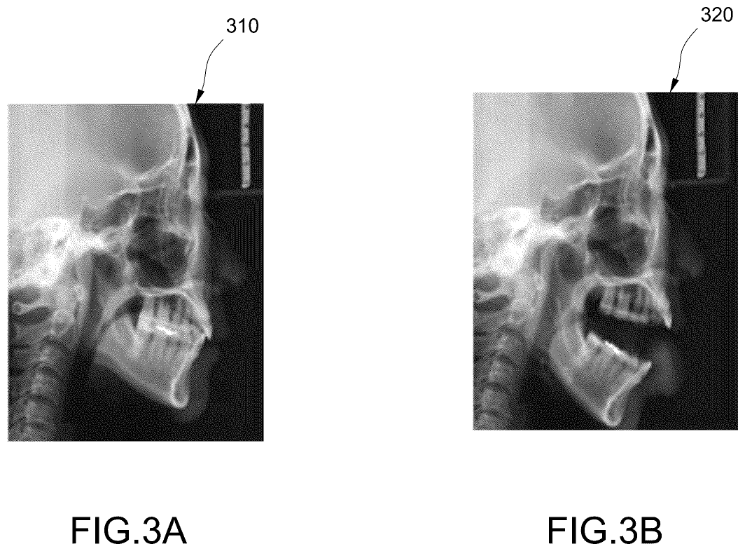

Referring to FIGS. 3A-3B, FIG. 3A shows a schematic diagram of a two-dimensional occlusion image 310 in the natural occlusion state, and FIG. 3B shows a schematic diagram of a two-dimensional occlusion image 320 in the maximum mouth-opening state. As shown in FIG. 3A, the two-dimensional occlusion image 310 is a side X-ray image of the patient's face and neck in the natural occlusion state. As shown in FIG. 3B, the two-dimensional occlusion image 320 is a side X-ray image of the patient's face and neck in the maximum mouth-opening state.

In some embodiments, the scanning modeling device 200 can be any type of external three-dimensional scanner used for scanning fields for three-dimensional reconstruction (e.g., contact probe three-dimensional scanners, non-contact optical three-dimensional scanners, or non-contact laser three-dimensional scanners). In some embodiments, the imaging device 300 can be any type of external photographic device used for medical purposes (e.g., ultrasound detection devices, computed tomography (CT) detection devices, magnetic resonance imaging (MRI) detection devices, or X-ray detection devices).

In some embodiments, the dental three-dimensional models can be three-dimensional models simulated by any three-dimensional modeling software (e.g., AutoCAD, Blender, or 3ds Max). In some embodiments, the two-dimensional occlusion images can be images used for medical purposes (e.g., ultrasound images, computed tomography (CT) images, magnetic resonance imaging (MRI) images, or X-ray images).

In some embodiments, the processor 120 generates a simulated airway in the occlusion target model. In some embodiments, the shape of the simulated airway is elliptical. In some embodiments, the area of the simulated airway is related to airflow, air fluid velocity, and air density. In some embodiments, airflow and air density can be pre-stored in memory 130. In some embodiments, the airflow (typically five to ten liters per minute) and air density (calculated based on pre-measured air temperature, pressure, and relative humidity) can be pre-stored in memory 130. In some embodiments, memory 130 stores a user-set simulated airway area, where the area of the simulated airway is inversely proportional to the air fluid velocity (the area of the simulated airway equals the ratio between air fluid velocity and airflow). In some embodiments, processor 120 generates a simulated airway in the occlusion target model based on this area. Consequently, the corresponding occlusion mold will have an airway corresponding to the simulated airway. It is noteworthy that a larger airway area results in a lower air fluid velocity (i.e., smoother breathing). Conversely, a smaller airway area results in a higher air fluid velocity (i.e., more rapid breathing).

In some embodiments, the processor 120 executes the simulation method described in the subsequent paragraphs to generate the occlusal splint target model. The transceiver circuit 110 then transmits the occlusal splint target model to the mold production machine 400, which produces an occlusal splint mold corresponding to the occlusal splint target model. In some embodiments, the mold production machine 400 can be a machine for molding molds (e.g., a 3D rubber jetting machine or an injection molding machine, etc.).

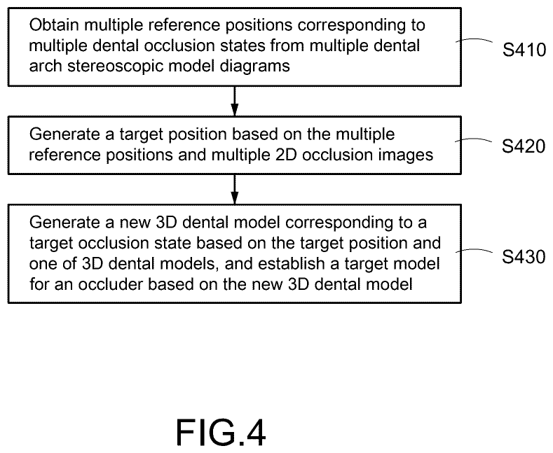

The simulation method of the present disclosure is further described below. Referring to FIG. 4, which illustrates a flowchart of the simulation method in some embodiments of the present disclosure, this simulation method is applicable to the occlusal splint mold simulation device 100 shown in FIG. 1.

As shown in FIG. 4, the simulation method includes steps S410 to S430. First, in step S410, the processor 120 obtains multiple reference positions corresponding to multiple dental occlusion states from the multiple dental arch stereoscopic model diagrams. In this embodiment, each of the reference positions represents the position of the dental arch center point of the mandible when the patient's oral cavity is in a corresponding dental occlusion state.

In some embodiments, the processor 120 establishes a dental arch coordinate system based on the position of the dental arch center point of the mandible in the stereoscopic model diagram of the natural occlusion state. Subsequently, the processor 120 obtains the position of the dental arch center point of the mandible in each of the multiple stereoscopic model diagrams based on the dental arch coordinate system as the multiple reference positions corresponding to the multiple dental occlusion states.

In some embodiments, the processor 120 sets the position of the dental arch center point of the mandible in the stereoscopic model diagram of the natural occlusion state as the origin of a dental arch coordinate system. Subsequently, the processor 120 sets the direction extending forward from the dental arch center point in each of the multiple stereoscopic model diagrams as the positive direction of the horizontal coordinate axis of the dental arch coordinate system (i.e., the direction of the line extending forward from the dental arch center point is set as the positive direction of the horizontal coordinate axis). Next, the processor 120 sets the direction extending downward from the dental arch center point in each of the multiple stereoscopic model diagrams as the positive direction of the vertical coordinate axis of the dental arch coordinate system (i.e., the direction of the line extending downward from the dental arch center point is set as the positive direction of the vertical coordinate axis).

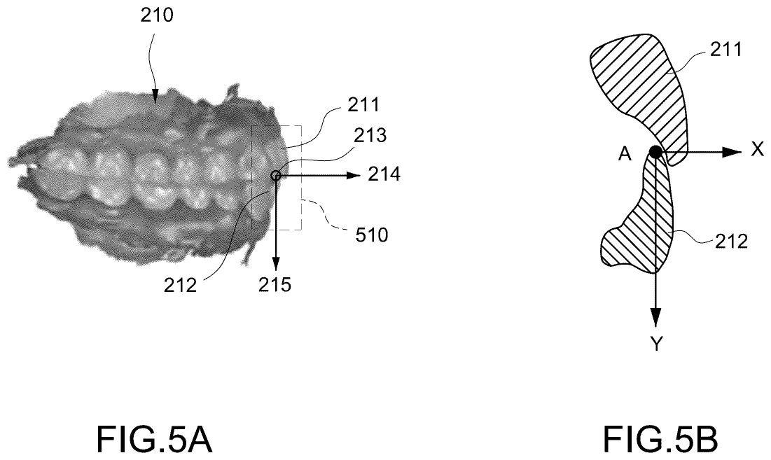

The acquisition of the reference positions is further explained with practical examples. Referring to FIGS. 5A and 5B, FIG. 5A illustrates a schematic diagram of the mandibular dental center point 213 in the 3D dental model 210 in the natural occlusion state in some embodiments of the present disclosure. FIG. 5B shows an enlarged side cross-sectional view of a portion 510 of the 3D dental model 210 in the natural occlusion state in some embodiments of the present disclosure. As shown in FIGS. 5A and 5B, the mandibular model 212 has a mandibular dental center point 213. The processor 120 takes the position A of the dental center point 213 as the origin of the dental coordinate system. Then, the processor 120 takes the direction of the forward extending line 214 from the dental center point 213 as the positive direction of the horizontal coordinate axis X of the dental coordinate system. Next, the processor 120 takes the direction of the downward extending line 215 from the dental center point 213 as the positive direction of the vertical coordinate axis Y of the dental coordinate system. Thus, the processor 120 can establish the dental coordinate system in the 3D dental model 210 in the natural occlusion state, and based on the dental coordinate system, take the position A as the reference position corresponding to the natural occlusion state (i.e., the coordinates of position A (i.e., the origin) in the dental coordinate system are taken as the coordinates of the reference position corresponding to the natural occlusion state).

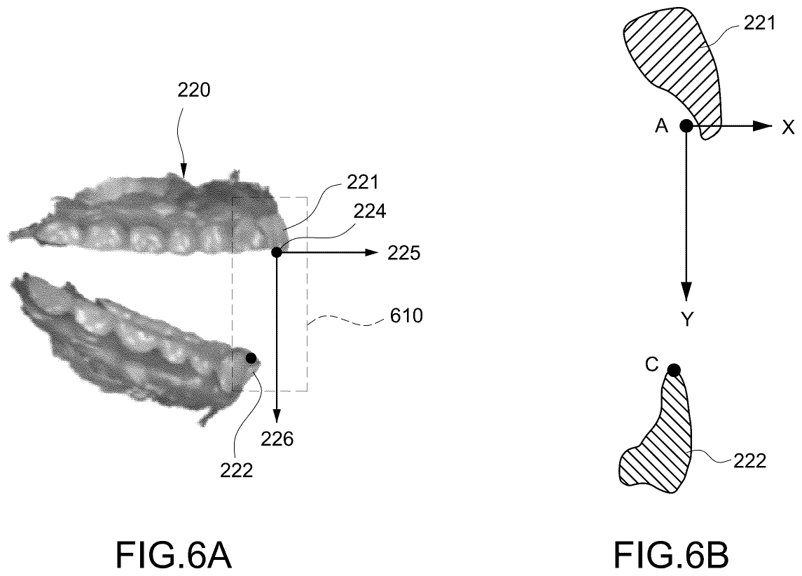

Referring to FIGS. 6A and 6B, FIG. 6A illustrates a schematic diagram of the mandibular dental center point 223 in the 3D dental model 220 in the maximum open mouth state in some embodiments of the present disclosure. FIG. 6B shows an enlarged side cross-sectional view of a portion 610 of the 3D dental model 220 in the maximum open mouth state in some embodiments of the present disclosure. As shown in FIGS. 6A and 6B, the processor 120 takes the position A of the dental center point 213 shown in FIG. 5B as the origin of the dental coordinate system in the 3D dental model 220. Then, the processor 120 takes the direction of the forward extending line 225 from the dental center point 223 as the positive direction of the horizontal coordinate axis X of the dental coordinate system. Next, the processor 120 takes the direction of the downward extending line 226 from the dental center point 223 as the positive direction of the vertical coordinate axis Y of the dental coordinate system. Thus, the processor 120 can establish the dental coordinate system in the 3D dental model 220 in the maximum open mouth state, and based on the dental coordinate system, take the position C on the mandibular model 222 as the reference position corresponding to the maximum open mouth state (i.e., the coordinates of position C in the dental coordinate system are taken as the coordinates of the reference position corresponding to the maximum open mouth state).

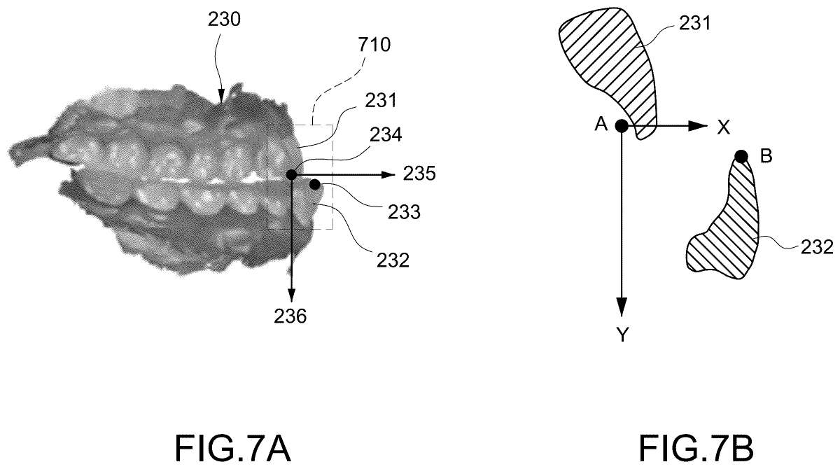

Referring to FIGS. 7A and 7B, FIG. 7A illustrates a schematic diagram of the mandibular dental center point 233 in the 3D dental model 230 in the maximum forward position state in some embodiments of the present disclosure. FIG. 7B shows an enlarged side cross-sectional view of a portion 710 of the 3D dental model 230 in the maximum forward position state in some embodiments of the present disclosure. As shown in FIGS. 7A and 7B, the processor 120 takes the position A of the dental center point 213 shown in FIG. 5B as the origin of the dental coordinate system in the 3D dental model 230. Then, the processor 120 takes the direction of the forward extending line 235 from the dental center point 233 as the positive direction of the horizontal coordinate axis X of the dental coordinate system. Next, the processor 120 takes the direction of the downward extending line 236 from the dental center point 233 as the positive direction of the vertical coordinate axis Y of the dental coordinate system. Thus, the processor 120 can establish the dental coordinate system in the 3D dental model 230 in the maximum forward position state, and based on the dental coordinate system, take the position B on the mandibular model 232 as the reference position corresponding to the maximum forward position state (i.e., the coordinates of position B in the dental coordinate system are taken as the coordinates of the reference position corresponding to the maximum forward position state).

Returning to FIG. 4, in step S420, the processor 120 generates a target position based on the multiple reference positions and the multiple 2D occlusion images. In this embodiment, the target position is the position of the mandibular dental center point when the patient's mouth is in the target occlusion state, where the target occlusion state indicates that the patient's epiglottis and trachea are in a more open state. In some embodiments, there is a proportional value between the opening width corresponding to the more open state and the opening width corresponding to the maximum open mouth state (which can be pre-set by the user), where the opening width is the width of the respiratory space formed by the patient's epiglottis and trachea. In some embodiments, the processor 120 recognizes the opening width corresponding to each of the multiple 2D occlusion images from the multiple 2D occlusion images, and generates the target position based on the multiple reference positions, the multiple 2D occlusion images, and the opening widths corresponding to each of the multiple 2D occlusion images. In some embodiments, the target position is the coordinates of the mandibular dental center point in the dental coordinate system in the target occlusion state.

In some embodiments, processor 120 can use any machine learning algorithm (e.g., YOLO (You Only Look Once) algorithm, Single Shot MultiBox Detector (SSD) algorithm, YOLACT (You Only Look At Coefficients) algorithm, Convolutional Neural Network (CNN) algorithm, Region-based Convolutional Neural Network (R-CNN) algorithm, Fast Region-based Convolutional Neural Network (Fast R-CNN) algorithm, Faster Region-based Convolutional Neural Network (Faster R-CNN) algorithm, or a combination thereof) to recognize the opening width corresponding to each of the multiple 2D occlusion images. For example, processor 120 can use multiple 2D occlusion images from other patients as samples and the corresponding opening widths as labels. Processor 120 can then use these samples and labels to train a recognition model with the YOLO algorithm, and use this trained model to identify the opening width for each of the multiple 2D occlusion images.

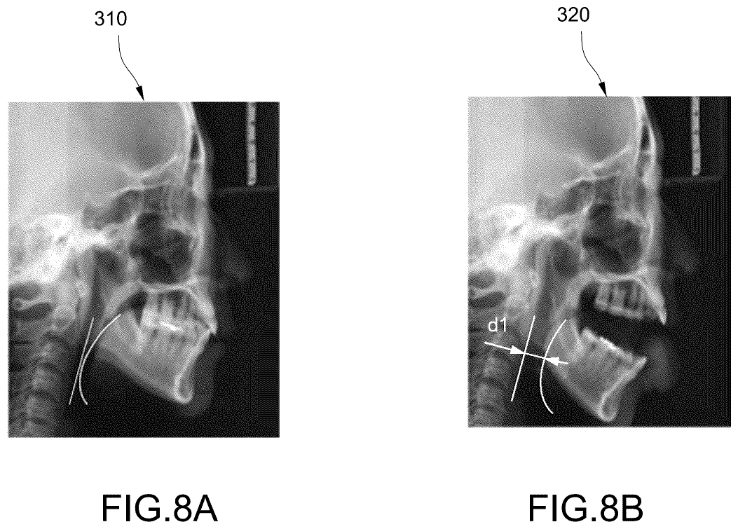

To illustrate the opening width with practical examples, refer to FIGS. 8A and 8B. FIG. 8A shows a schematic diagram of the opening in a 2D occlusion image 310 corresponding to the natural occlusion state in some embodiments. FIG. 8B shows a schematic diagram of the opening in a 2D occlusion image 320 corresponding to the maximum open mouth state in some embodiments. As shown in FIG. 8A, when the patient's mouth is in the natural occlusion state, the opening of the epiglottis and trachea is fully closed (i.e., a fully closed state). At this time, processor 120 identifies the opening width corresponding to 2D occlusion image 310 as zero. As shown in FIG. 8B, when the patient's mouth is in the maximum open mouth state, the opening of the epiglottis and trachea is fully open (i.e., maximum opening state). At this time, processor 120 identifies the opening width corresponding to 2D occlusion image 320 as the maximum width d1.

In some embodiments, processor 120 can use any machine learning algorithm to generate the target position of the dental coordinate system based on multiple reference positions, multiple 2D occlusion images, and the corresponding opening widths of these images. For example, processor 120 can use multiple reference positions, multiple 2D occlusion images from other patients, and the corresponding opening widths as samples, and the pre-measured target positions of the dental coordinate system for other patients as labels. Processor 120 can then use these samples and labels for embedding processing (i.e., vectorizing all samples and labels) and train another recognition model with a Convolutional Neural Network (CNN) algorithm. This allows processor 120 to use this model to generate the target position of the dental coordinate system for the target patient based on their reference positions, 2D occlusion images, and the corresponding opening widths.

Returning to FIG. 4, in step S430, processor 120 generates a new 3D dental model corresponding to the target occlusion state based on the target position and one of the 3D dental models, and establishes a target model for the occluder based on the new 3D dental model. In some embodiments, the 3D dental model can be any of the multiple 3D dental models (e.g., the 3D dental model in the natural occlusion state). In some embodiments, processor 120 adjusts the position of the mandibular model in the selected 3D dental model so that the dental center point in the mandibular model aligns with the target position, thereby generating a new 3D dental model corresponding to the target occlusion state. Processor 120 then establishes the target model for the occluder based on the newly generated 3D dental model.

In other words, processor 120 adjusts the 3D dental model to match the shape of the patient's mouth in the target occlusion state. This adjusted shape is used as a template for the occluder target model, leading to the creation of the optimal occluder model. Finally, processor 120 sends this occluder target model to the mold production machine 400, which then produces an occluder mold corresponding to this target model. It is worth noting that when the patient uses this occluder mold, they will experience the optimal therapeutic effect. Specifically, the width of the airway formed by the patient's epiglottis and trachea will be at a moderate opening width (i.e., a slightly open state), and the patient's jaw will not feel sore due to excessive force.

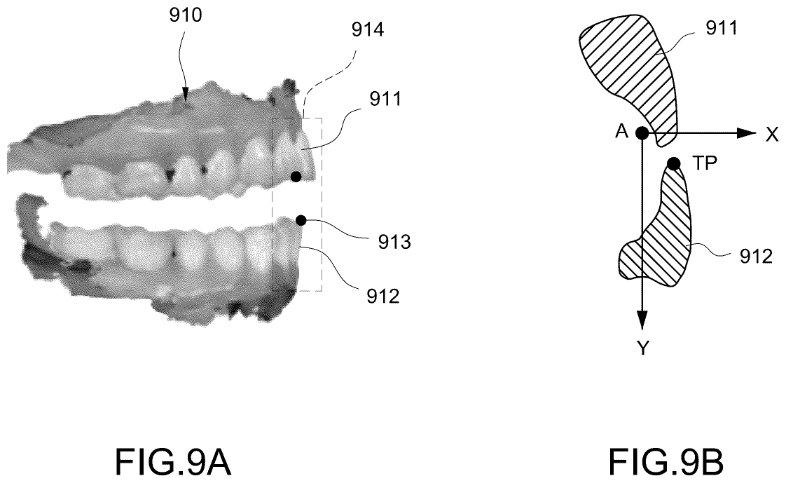

To illustrate the target position and the new 3D dental model, refer to FIGS. 9A and 9B. FIG. 9A shows a schematic of the new 3D dental model 910 in the target occlusion state, and FIG. 9B shows an enlarged side cross-sectional view of a portion 914 of the new 3D dental model 910. As illustrated in FIGS. 5A and 9A, assuming the 3D dental model is model 210, processor 120 adjusts the position of the mandibular model 212 in model 210 until the dental center point 213 aligns with the target position.

Furthermore, as shown in FIGS. 5B and 9B, based on the dental coordinate system established in 3D dental model 210, processor 120 can move the position of the mandibular dental center point 213 to align with the target position TP, thus generating the new 3D dental model 910. This displacement results in the new 3D dental model 910, where the maxillary model 911's position remains the same as in 3D dental model 210, and the mandibular model 912's position aligns with the target position TP.

Although using the occluder mold produced from the new 3D dental model can solve obstructive sleep apnea issues, the patient may not always find the mold comfortable. Therefore, after obtaining the adjustment parameters for each patient, this disclosure further adjusts the new 3D dental model to create an adjusted 3D dental model, ensuring that the final produced occluder mold provides both “improved obstructive sleep apnea” and “comfortable occlusion” effects. In some embodiments, processor 120 adjusts the position of the mandibular model in the new 3D dental model based on the movement parameters to produce an adjusted 3D dental model, and establishes the occluder target model for the adjusted 3D dental model. In some embodiments, the movement parameters indicate the horizontal advancement ratio of the mandibular dental center point (e.g., 100% corresponds to the lateral coordinate of the mandibular dental center point in the maximum forward advancement state, and 0% corresponds to the lateral coordinate in the target occlusion state). Through such adjustments, processor 120 generates the adjusted occluder target model. The user can then try the occluder mold produced based on the adjusted target model. If the patient feels comfortable, this mold is the most suitable for the patient. When using this mold to improve obstructive sleep apnea, the patient's jaw will be in the most comfortable state.

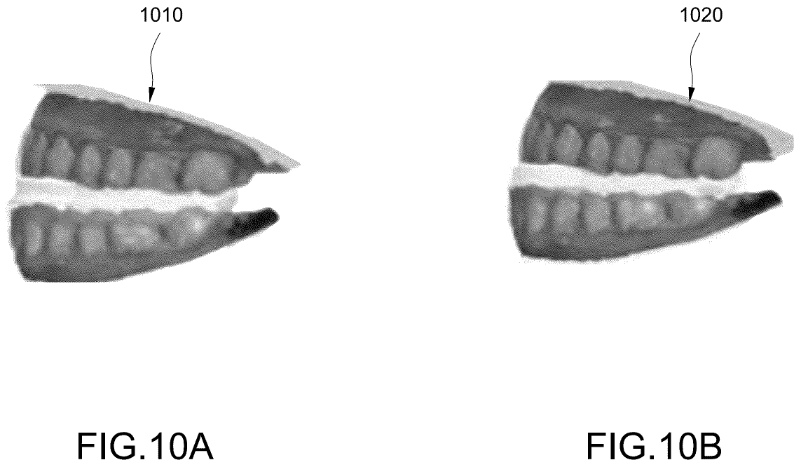

To illustrate the movement parameters, refer to FIGS. 10A and 10B. FIG. 10A shows the adjusted 3D dental model 1010, and FIG. 10B shows another adjusted 3D dental model 1020. Assuming the horizontal advancement ratio corresponding to the lateral coordinate of the mandibular dental center point in the maximum forward advancement state is 100%, and the ratio in the target occlusion state is 0%, in the example of FIG. 10A, the displacement parameter for the adjusted 3D dental model 1010 is 60%, while in the example of FIG. 10B, the displacement parameter for the adjusted 3D dental model 1020 is 70%.

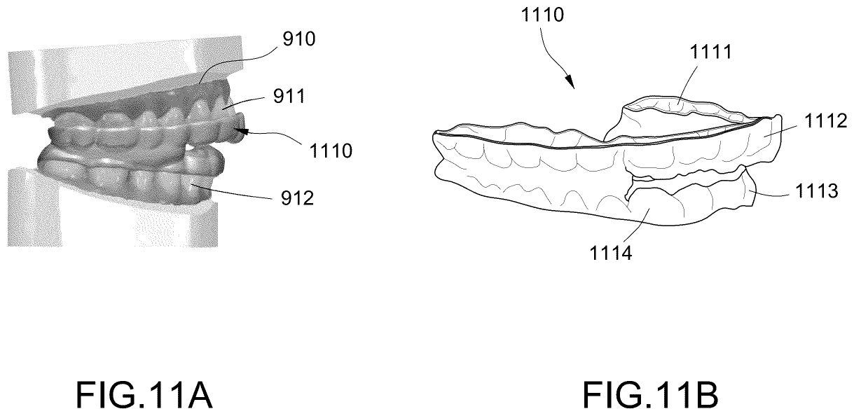

To illustrate the occluder target model, refer to FIGS. 11A and 11B. FIG. 11A shows a schematic of the occluder target model 1110, while FIG. 11B depicts the detailed structure of the occluder target model 1110. As shown in FIG. 11A, processor 120 generates an occluder target model 1110 between the maxillary model 911 and mandibular model 912 in the new 3D dental model 910. FIG. 11B shows that the occluder target model 1110 includes an upper arch 1112 and a lower arch 1113, which are connected at both ends. The upper arch 1112 has a maxillary dental engagement groove 1111 that matches the shape of the maxillary model 911, and the lower arch 1113 has a mandibular dental engagement groove 1114 that matches the shape of the mandibular model 912. The maxillary dental engagement groove 1111 and the mandibular dental engagement groove 1114 are positioned to face away from each other. When the patient wears the occluder mold corresponding to the occluder target model 1110, the grooves in the occluder mold that match the maxillary dental engagement groove 1111 and the mandibular dental engagement groove 1114 will engage with the patient's maxillary and mandibular dental arches, respectively, fixing the relative position of the patient's maxillary and mandibular arches to the target position.

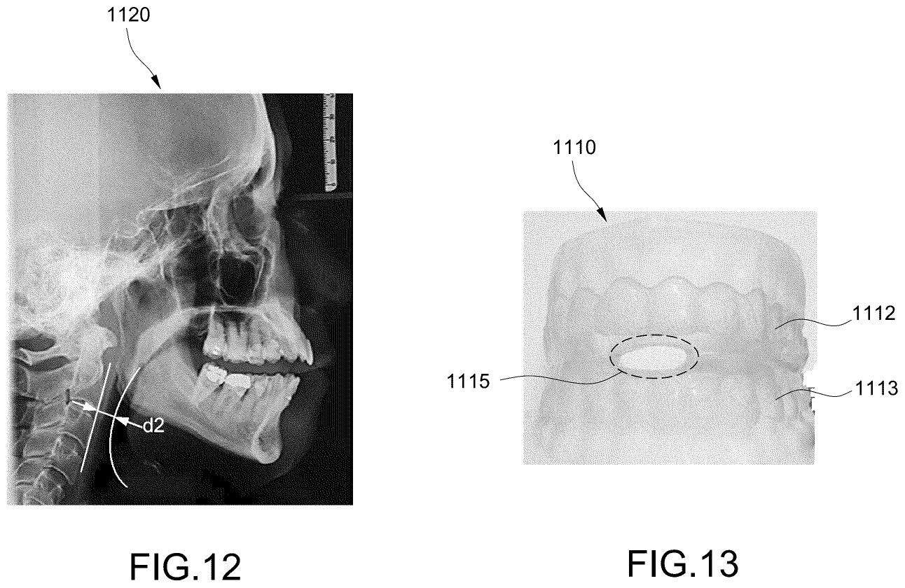

To illustrate the 2D occlusion image obtained from photographing a patient wearing the occluder mold, refer to FIG. 12. FIG. 12 shows a schematic of the 2D occlusion image 1120 taken from a patient wearing the occluder mold. Processor 120 can identify the target width d2 from the 2D occlusion image 1120, where the target width d2 represents the width of the airway formed by the epiglottis and trachea when the patient's mouth is in the target occlusion state. Compared to the maximum width d1 shown in FIG. 8B, the target width d2 may be two-thirds of the maximum width d1.

In some embodiments, processor 120 creates a simulated airway opening in the occluder target model. The shape of the simulated airway opening may be elliptical in some embodiments. The area of the simulated airway opening is related to airflow, air velocity, and air density. Airflow and air density can be pre-stored in memory 130. For example, airflow (typically five to ten liters per minute) and air density (calculated based on pre-measured temperature, pressure, and relative humidity) can be stored in memory 130. In some embodiments, memory 130 stores a user-defined area for the simulated airway opening, where the area is inversely proportional to the air velocity (the area of the simulated airway opening equals the ratio between air velocity and airflow). Processor 120 generates the simulated airway opening in the occluder target model based on this area. Consequently, the corresponding occluder mold will have an airway opening corresponding to the simulated airway opening. It is important to note that a larger airway opening area results in a lower air velocity (i.e., smoother breathing), while a smaller airway opening area results in a higher air velocity (i.e., more rapid breathing).

To illustrate the simulated airway opening, refer to FIG. 13. FIG. 13 shows a schematic of the simulated airway opening 1115 in some embodiments. The simulated airway opening 1115, shaped elliptically, is located at the junction between the upper arch 1112 and lower arch 1113 in the occluder target model 1110.

In summary, the disclosed simulation device and method for occluder molds use scanned 3D dental models and 2D occlusion images from multiple occlusion states of the patient's teeth to determine the position of the mandibular dental center point in the target occlusion state. Additionally, the method generates the optimal occluder model based on this position and one of the 3D dental models to produce a mold that best fits the patient. As a result, the patient will not only experience improved breathing by having the epiglottis and trachea in a more open state (thus alleviating obstructive sleep apnea) but also maintain the most comfortable jaw position. This avoids traditional surgical treatments, reducing medical risks and postoperative complications. Furthermore, the method allows for the generation of a simulated airway opening in the optimal occluder model, ensuring more comfortable breathing for the patient when using the mold.

While this disclosure has been described by means of specific embodiments, numerous modifications and variations may be made thereto by those skilled in the art without departing from the scope and spirit of this disclosure set forth in the claims.

What is claimed is:

1. An occlusal mold simulation device, adapted for simulating an occlusal mold in a three-dimensional virtual space, wherein the simulation device comprises:
a transceiver circuit, configured to receive from a scanning and modeling device a plurality of tooth arch 3D models corresponding to various dental occlusion states in the three-dimensional virtual space, and to receive from an imaging device a plurality of two-dimensional occlusion images corresponding to the various dental occlusion states, wherein the various dental occlusion states comprise a maximum forward protrusion state of the mandible, a maximum mouth opening state, and a natural occlusion state;
a memory, configured to store a plurality of instructions;
a processor, connected to the transceiver circuit and the memory, and configured to execute the plurality of instructions to perform the following steps:
obtain a plurality of reference positions from the plurality of tooth arch 3D models, wherein each of the reference positions corresponds to a tooth arch center point of the mandible when the patient's oral cavity is in the respective dental occlusion state corresponding to the reference position;
generate a target position based on the plurality of reference positions and the plurality of two-dimensional occlusion images, wherein the target position corresponds to a tooth arch center point of the mandible when the patient's oral cavity is in a target occlusion state, wherein the target occlusion state indicates that the patient's epiglottis and trachea are in a more open state; and
generate a new tooth arch 3D model corresponding to the target occlusion state based on the target position and establish a target model for the occlusal mold.

2. The occlusal mold simulation device according to claim 1, wherein the maximum forward protrusion state of the mandible is a state in which the mandible of the patient moves forward a maximum horizontal distance along a direction parallel to the ground, the maximum mouth opening state is a state in which the mandible of the patient moves downward a maximum vertical distance along a direction perpendicular to the ground, and the natural occlusion state is a state in which the mandible of the patient naturally occludes with the maxilla.

3. The occlusal mold simulation device according to claim 1, wherein the plurality of tooth arch 3D models in the three-dimensional virtual space are generated by scanning the patient's oral cavity in the various dental occlusion states using the scanning and modeling device, and the plurality of two-dimensional occlusion images are obtained by capturing the patient's facial and neck images in the various dental occlusion states using the imaging device.

4. The occlusal mold simulation device according to claim 1, wherein in the step of generating the target position based on the plurality of reference positions and the plurality of two-dimensional occlusion images, the processor is configured to:
identify an opening width corresponding to each of the plurality of two-dimensional occlusion images from the plurality of two-dimensional occlusion images, and generate the target position based on the plurality of reference positions, the plurality of two-dimensional occlusion images, and the opening width corresponding to each of the two-dimensional occlusion images, wherein the opening width is a width of an airway formed by the patient's epiglottis and trachea.

5. The occlusal mold simulation device according to claim 1, wherein in the step of generating the new tooth arch 3D model corresponding to the target occlusion state based on the target position, the processor is configured to:
adjust the position of a mandible model in one of the plurality of tooth arch 3D models to align the tooth arch center point of the mandible in the tooth arch 3D model with the target position, thereby generating the new tooth arch 3D model corresponding to the target occlusion state.

6. A method for simulating an occlusal mold, adapted for simulating an occlusal mold in a three-dimensional virtual space, wherein the simulation method comprises:
using a processor to obtain a plurality of reference positions from the plurality of tooth arch 3D models in the three-dimensional virtual space, wherein each reference position corresponds to a tooth arch center point of the mandible when the patient's oral cavity is in the respective dental occlusion state corresponding to the reference position, wherein the plurality of tooth arch 3D models and the plurality of two-dimensional occlusion images correspond to various dental occlusion states, comprising a maximum forward protrusion state of the mandible, a maximum mouth opening state, and a natural occlusion state;
using the processor to generate a target position based on the plurality of reference positions and the plurality of two-dimensional occlusion images, wherein the plurality of two-dimensional occlusion images correspond to the various dental occlusion states, wherein the target position corresponds to a tooth arch center point of the mandible when the patient's oral cavity is in a target occlusion state, wherein the target occlusion state indicates that the patient's epiglottis and trachea are in a more open state; and
using the processor to generate a new tooth arch 3D model corresponding to the target occlusion state based on the target position, and establishing a target model for the occlusal mold.

7. The method for simulating an occlusal mold according to claim 6, wherein the maximum forward protrusion state of the mandible is a state in which the mandible of the patient moves forward a maximum horizontal distance along a direction parallel to the ground, the maximum mouth opening state is a state in which the mandible of the patient moves downward a maximum vertical distance along a direction perpendicular to the ground, and the natural occlusion state is a state in which the mandible of the patient naturally occludes with the maxilla.

8. The method for simulating an occlusal mold according to claim 6, wherein the plurality of tooth arch 3D models in the three-dimensional virtual space are generated by scanning the patient's oral cavity in the various dental occlusion states using a scanning and modeling device, and the plurality of two-dimensional occlusion images are obtained by capturing the patient's facial and neck images in the various dental occlusion states using an imaging device.

9. The method for simulating an occlusal mold according to claim 6, wherein the step of generating the target position based on the plurality of reference positions and the plurality of two-dimensional occlusion images comprises:
using the processor to identify an opening width corresponding to each of the plurality of two-dimensional occlusion images from the plurality of two-dimensional occlusion images, and generating the target position based on the plurality of reference positions, the plurality of two-dimensional occlusion images, and the opening width corresponding to each of the two-dimensional occlusion images, wherein the opening width is a width of an airway formed by the patient's epiglottis and trachea.

10. The method for simulating an occlusal mold according to claim 6, wherein the step of generating the new tooth arch 3D model corresponding to the target occlusion state comprises:
using the processor to adjust the position of a mandible model in one of the plurality of tooth arch 3D models to align the tooth arch center point of the mandible in the tooth arch 3D model with the target position, thereby generating the new tooth arch 3D model corresponding to the target occlusion state.
